(19) United States
(12) Patent Application Publication
(10) Pub. No.: US 2011/0319979 A1
(43) Pub. Date: Dec. 29, 2011

(54) STENT AND METHOD OF MOUNTING THE SAME

(75) Inventors: Soo Won SEO, Seongnam-si (KR); In Wook Choo, Seoul (KR); Jae Jun Kim, Seoul (KR); Jin Yong Kim, Seoul (KR); Hong Suk Park, Seoul (KR)

(73) Assignee: SAMSUNG LIFE WELFARE FOUNDATION, Seoul (KR)

(21) Appl. No.: 13/167,802

(22) Filed: Jun. 24, 2011

(30) Foreign Application Priority Data
Jun. 25, 2010 (KR) 10-2010-0060742

(51) Int. Cl. A61F 2/82; B23P 11/02

(52) U.S. Cl. 623/1.16; 29/446

(57) ABSTRACT

A stent that maintains flexibility in a length direction and prevents fatigue failure by securing predetermined durability is disclosed.

The stent may includes a plurality of cylindrical members having a hollow cylindrical shape with both ends open, and adapted to decrease a diameter thereof by an external force so as to be mounted in a tubule, and a connecting member connecting cylindrical members confronting each other.

### CROSS-REFERENCE TO RELATED APPLICATION

This application claims priority to and the benefit of Korean Patent Application No. 10-2010-0060742 filed in the Korean Intellectual Property Office on Jun. 25, 2010, the entire contents of which are incorporated herein by reference.

### BACKGROUND OF THE INVENTION

(a) Field of the Invention

The present invention relates to a stent. More particularly, the present invention relates to a stent that maintains flexibility in a length direction and prevents fatigue failure by securing predetermined durability.

(b) Description of the Related Art

Generally, tubules (lumen or arteries) in a human body become narrow due to disease, injury, operations, and so on. If the tubules become narrow, functions thereof may be deteriorated, and the tubules may not operate normally in extreme cases. In these cases, various devices for expanding the narrowed tubules or not allowing the tubules to narrow are used. A stent is a typical device for expanding narrowed tubules.

If the tubules become narrow, for example when narrowness of the esophagus occurs due to cancer of the esophagus, blood does not circulate smoothly due to hardening of the arteries, or narrowness of a track through which bile from the liver passes occurs, the stent is mounted at the narrowed tubule so as to expand the tubule and to maintain the expanded state. Thereby, food, blood, or bile can flow smoothly.

Such stents have different structures and characteristics according to a method by which the stent is mounted as well as positions and types of the tubules.

In a case that a stent is mounted at a digestive track for example, a stent having flexibility in a length direction is used so as to be flexible in response to movements of the digestive track. In the past, the stent has been mounted in the tubules by using X-ray vision medical equipment. Since an insertion device of an endoscope, has recently become narrow, the stent can be mounted through a sine channel of the endoscope. In this case, a stent that is not dressed can be used considering an interior diameter of the insertion device.

As described above, different types of stents can be used according to positions and types of the tubules and mounting method of the stent. There are various criteria for selecting the stent, such as flexibility in the length direction, extendibility, thickness, durability, and so on.

The flexibility in the length direction and the durability are the most important criteria to a patient in which the stent is mounted. The flexibility in the length direction is important in that the patient can feel a sense of difference due to insertion of the stent, and the durability is important in that it determines the replacement period of the stent.

In a case that the flexibility in the length direction is enhanced, however, dynamic fatigue of the stent can increase and durability of the stent may be deteriorated. In a case that the durability, on the contrary, is increased, rigidity due to material and structure of the stent may be increased and the flexibility to the length direction may be reduced. That is, the flexibility to the length direction and the durability have an inverse relationship.

Therefore, a stent satisfying two important criteria to the patient should be developed.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a stent having advantages of preventing fatigue failure by maintaining flexibility in a length direction and securing predetermined durability.

Technical objects of the present invention are not limited to the technical objects described in this specification, and technical objects that are not described can be understood by a person skilled in the art referring to the following description.

A stent according to exemplary embodiments of the present invention includes: a plurality of cylindrical members having a hollow cylindrical shape having both ends open, and adapted to decrease a diameter thereof by an external force so as to be mounted in a tubule; and a connecting member connecting cylindrical members confronting each other.

In one or some embodiments, the cylindrical member is manufactured by disposing one or more lines in a zigzag shape on an external circumference thereof so as to form a plurality of peak parts and valley parts.

In one or some embodiments, the connecting member connects the closest peak part and valley part of confronting cylindrical members.

In one or some embodiments, the connecting member is manufactured by a linear thin member or a ring-shaped thin member.

In one or some embodiments, the connecting member is only disposed on one semicircular portion in a case that the cylindrical member is divided by a plane passing through the center of the cylindrical member.

In one or some embodiments, the connecting member is disposed in one row.

In one or some embodiments, the row of the connecting member has a linear shape.

In one or some embodiments, the row of the connecting member has a zigzag shape.

In one or some embodiments, the connecting members are disposed in two or more rows.

In one or some embodiments, the row of the connecting member has a linear shape, a zigzag shape, or a combination of a linear shape and a zigzag shape.

In one or some embodiments, the cylindrical member is made of a harmless metal or resin material.

In one or some embodiments, the connecting member is made of a harmless metal or resin material.

In one or some embodiments, the cylindrical member is adapted to change a diameter thereof by changing angles of the peak part and the valley part in a case that the external force is applied to the cylindrical member.

According to exemplary embodiments of the present invention, a method of mounting a stent having a plurality of cylindrical members adapted to decrease a diameter thereof by an external force, and a connecting member connecting cylindrical members confronting each other is disclosed.

The method includes: decreasing the diameter of the plurality of cylindrical members by exerting the external force thereon; inserting the cylindrical members in a tubule; mounting one of the cylindrical members at the tubule by removing the external force exerted on the one of the cylindrical members; and sequentially mounting the others of the cylindrical members.

In one or more embodiments, the cylindrical members are mounted in a sequence from the cylindrical member inserted deepest to the cylindrical member inserted shallowest.

In one or more embodiments, the cylindrical members are mounted in a sequence from the cylindrical member inserted shallowest to the cylindrical member inserted deepest.

In one or more embodiments, the connecting member is adapted to move the plurality of cylindrical members relatively according to a movement of the tubule.

### BRIEF DESCRIPTION OF THE DRAWINGS

FIG. 1 to FIG. 5 are top plan views of a stent according to various exemplary embodiments of the present invention.

FIG. 6 and FIG. 7 are top plan views of a stent according to exemplary embodiments of the present invention for showing an operation of the stent.

FIG. 8 is a top plan view of a stent according to another exemplary embodiment of the present invention for showing an operation of the stent.

### DESCRIPTION OF SYMBOLS

100: cylindrical member
200: connecting member

### DETAILED DESCRIPTION OF THE EMBODIMENTS

Hereinafter, exemplary embodiments of the present invention will be described in detail referring to the drawings. In explaining exemplary embodiments of the present invention, well-known functions or structures can be omitted for clearly describing the present invention.

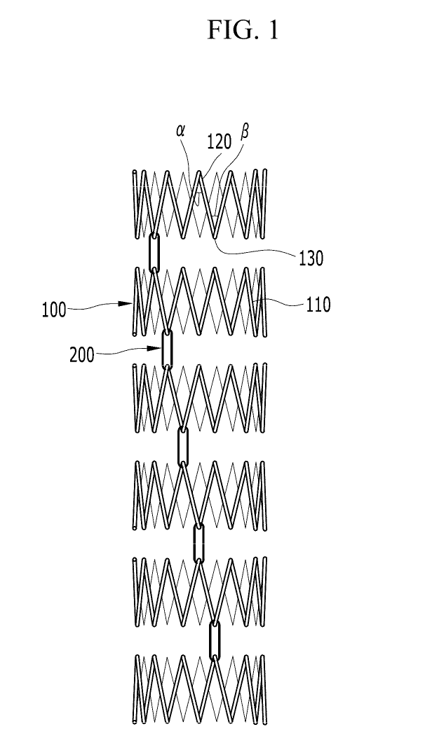

Referring to FIG. 1 to FIG. 5, a stent according to exemplary embodiments of the present invention will be described in detail.

Herein, FIG. 1 to FIG. 5 are top plan views of a stent according to various exemplary embodiments of the present invention.

As shown in FIG. 1 to FIG. 5, a stent according to exemplary embodiments of the present invention includes a plurality of cylindrical members 100 and at least one connecting member 200 connecting adjacent cylindrical members 100.

In one or some embodiments, the plurality of cylindrical members 100 are disposed linearly or non-linearly. The cylindrical member 100 forms an external circumference of the stent according to exemplary embodiments of the present invention, and is a hollow member.

Herein, the external circumference of the cylindrical member 100 contacts the interior circumference of tubules. An inner space of the cylindrical member 100, that is, a diameter of the cylindrical member 100, is adapted to be reduced when an external force stronger than a predetermined strength is applied to the cylindrical member 100.

Herein, the external force is a force applied to the cylindrical member 100 for easily inserting the stent according to exemplary embodiments of the present invention into the tubules. If the larger external force than the predetermined strength is applied to the cylindrical member 100, the diameter of the cylindrical member 100 is decreased and the stent can be easily inserted in the tubules. In addition, the larger external force than the predetermined strength is a force that can reduce the diameter of the cylindrical member 100 sufficiently when a device for inserting the stent applies force to the cylindrical member 100. The larger external force than the predetermined strength can be determined according to structures and materials of the cylindrical members 100.

Referring to FIG. 1 to FIG. 3, the cylindrical member 100 will be described in further detail.

As shown in FIG. 1 to FIG. 3, the cylindrical member 100 is formed by one or a plurality of lines 110. The cylindrical member 100 is formed by forming a closed curve by connecting both ends of the line 110 and disposing the closed curve along a length direction of the cylindrical member 100 so as to have a predetermined length. The closed curve is disposed on the external circumference of the cylindrical member 100. The line 110 has a predetermined thickness. The predetermined thickness can be determined by a person of ordinary skill in the art considering target strength, material, and disposition of the closed curve.

As shown in FIG. 1, the cylindrical member 100 can be manufactured by disposing the closed curve formed by one line 110 on the external circumference thereof. The closed curve can be disposed in a zigzag shape having a plurality of peak parts 120 and valley parts 130. Herein, the peak part 120 is a convex part along the length direction of the cylindrical member 100 (i.e., upwardly convex in the drawings), and the valley part 130 is a concave part along the length direction of the cylindrical member 100 (i.e., downwardly convex in the drawings). Angles α and β of the peak part 120 and the valley part 130 are changed as the external force is exerted thereon. That is, in a case that the external force is exerted on the cylindrical member 100, the angles α and β of the peak part 120 and the valley part 130 are decreased and the diameter of the cylindrical member 100 is also decreased. In a case that the angles α and β of the peak part 120 and the valley part 130 are increased by the external force, the diameter of the cylindrical member 100 is also increased. If the external force having been applied to the cylindrical member 100 vanishes, the cylindrical member 100 returns to an original shape thereof. That is, the cylindrical member 100 has elasticity.

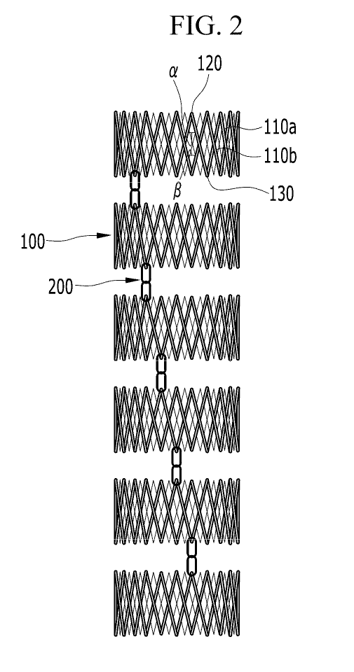

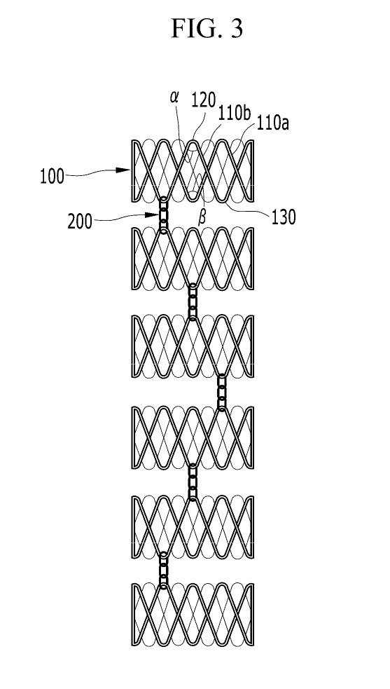

As shown in FIG. 2 and FIG. 3, the cylindrical member 100 is manufactured by disposing the closed curves formed by two lines 110a and 110b on the external circumference thereof. At this time, the closed curves are disposed in the same shape or different shapes. In addition, the closed curves are disposed circumferentially with a predetermined distance therebetween. At this time, the closed curves are connected to or contact each other at a crossing point therebetween. Each closed curve is disposed in a zigzag shape having a plurality of peak parts 120 and valley parts 130. As shown in FIG. 2, the peak part 120 and the valley part 130 have sharp ends. In addition, as shown in FIG. 3, the peak part 120 and the valley part 130 have blunt ends. The angles α and β of the peak part 120 and the valley part 130 are changed as the external force is exerted thereon. The diameter of the cylindrical member 100 increases or decreases by the change of the angles α and β of the peak part 120 and the valley part 130.

Dimensions of the cylindrical member 100 are changed according to a position and a type of the tubule in which the stent is mounted. The length and the diameter of the cylindrical member 100 are controlled according to the disposition of the closed curves. The dispositions of the closed curves are not limited to those described in this specification. It is to be understood that dispositions of the closed curves that can change the diameter of the cylindrical member 100 by the external force are included in the scope of the present invention.

Meanwhile, both ends of the cylindrical member 100 are open so that food, blood, or bile can pass therethrough.

In addition, the material of the cylindrical member 100 can be changed according to positions and types of the tubules. In one or some embodiments, the cylindrical member 100 is made of a metal material or a resin material so as to secure durability and strength. A harmless metal material or resin material is preferable.

One or a plurality of cylindrical members 100, as described above, are disposed so as to form one row. Compared with the case that one cylindrical member 100 is used, the position of the stent can be easily controlled in the tubule if a plurality of cylindrical members 100 is used.

That is, in a case that one cylindrical member 100 is used, the stent is mounted in the tubule at one time. Therefore, a mounting position of the stent should be carefully controlled in initial insertion of the stent. In a case that a plurality of cylindrical members 100 are used, they are mounted in the tubule sequentially. Therefore, the position of each cylindrical member 100 can be easily controlled.

The plurality of cylindrical members 100 are connected to each other by at least one connecting member 200.

The connecting member 200 connects adjacent cylindrical members 100 such that the stent has a pipe shape. The cylindrical members 100 can move relative to each other by the connecting member 200. That is, the connecting member 200 enables the shape of the stent to be controlled according to the shape of the tubule.

In one or some embodiments, the connecting member 200 is manufactured with thin members.

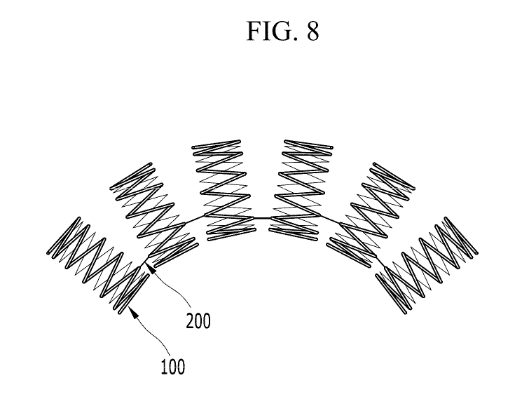

In one or some embodiments, a linear connecting member 200 can be used as shown in FIG. 8. In this case, one end of the connecting member 200 is connected to one cylindrical member 100 and the other end of the connecting member 200 is connected to another cylindrical member 100 so as to connect the adjacent cylindrical members 100.

In one or some embodiments, a ring-shape thin member is used as the connecting member 200, as shown in FIG. 1 to FIG. 7.

Herein, the ring-shape thin member means a thin member having a closed curve shape by connecting both ends of the thin member to each other as shown in FIG. 1. In one or more embodiments, the ring-shape thin member is manufactured by cutting a thin metal pipe perpendicularly or slantedly to a length direction thereof.

In one or some embodiments, a plurality of ring-shape thin members are connected to each other and used as one connecting member 200. Herein, a plurality of ring-shape thin members can be connected by passing one ring-shape thin member through a space formed by another ring-shape thin member or by twisting one ring-shape thin member so as to join a plurality of ring-shape thin members.

In a case that the connecting member 200 of linear thin member is used, manufacturing processes are simplified. On the contrary, in a case that the connecting member 200 of a ring-shape thin member is used, durability is improved.

In one or some embodiments, the connecting member 200 is adapted to connect the closest peak part 120 and valley part 130 of the confronting cylindrical members 100.

The connecting member 200 is made of a metal material or a resin material considering durability thereof. A harmless metal material or resin material is preferable.

In one or some embodiments, the connecting member 200 is made of an elastic material such that the connecting member 200 can elongate sufficiently in the length direction of the cylindrical member 100. In a case that the connecting member 200 of a ring-shape thin member is used, the connecting member 200 can elongate in the length direction of the cylindrical member 100 to a certain degree due to shape features. In a case that the connecting member 200 of linear thin member is used, the connecting member 200 can elongate in the length direction of the cylindrical member 100 due to material characteristics.

In one or more embodiments, the connecting member 200 is made of a rigid material pro re nata.

In a case that the connecting member 200 can elongate or be shortened in the length direction of the cylindrical member 100, sequential installation of the stent can be simplified. That is, after one cylindrical member 100 is mounted at a desired position, a mounting position of another cylindrical member 100 can be easily controlled.

Since the connecting member 200 connects the cylindrical members 100, the connecting member 200 can be connected to any peak part 120 and valley part 130 of the cylindrical members 100.

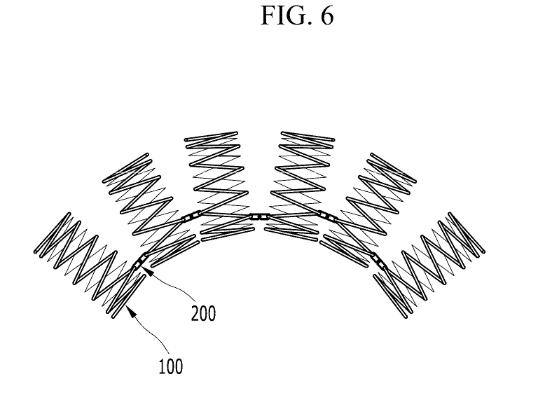

For example, the connecting members 200 are disposed only on one semicircular portion in a case that the cylindrical member 100 is divided by a plane passing through the center of the cylindrical member 100, as shown in FIG. 6. In a case that the connecting member 200 is positioned on one semicircular portion, the shape of the stent can be easily controlled.

On the contrary, some of the connecting members 200 are positioned on one semicircular portion and the rest of the connecting members 200 are positioned on the other semicircular portion. In this case, the stent can be easily mounted by restricting free movements of the cylindrical members 100.

In the drawings, a bold line denoting the cylindrical member 100 represents the cylindrical member 100 positioned on one semicircular portion, and a thin line denoting the cylindrical member 100 represents the cylindrical member 100 positioned on the other semicircular portion.

Positions of the connecting members 200 are determined by a designer considering type and movement of the tubules in which the connecting members 200 are mounted, and mounting restrictions. For example, because the designer can predict the movement of the tubule in which the stent is mounted, the connecting member 200 is disposed not at a relaxing portion of the tubule but at a contracting portion of the tubule considering the movement of the tubule. Thereby, patients cannot feel uncomfortable due to implantation of the stent.

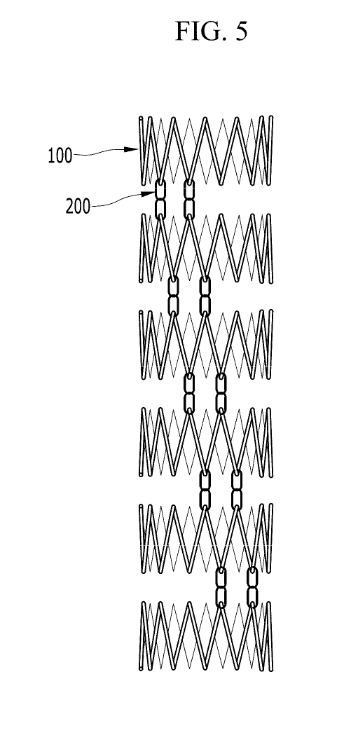

In one or some embodiments, the connecting members 200 are disposed in one row (referring to FIG. 1 to FIG. 4). In one or some embodiments, the connecting members 200 are disposed in two or more rows (referring to FIG. 5).

The number of rows in which the connecting members 200 are disposed can be determined by the designer according to the type and the position of the tubule in which the stent is mounted. That is, if flexibility in the length direction is a more important criterion than durability, the connecting members 200 are disposed in one row. On the contrary, if durability is a more important criterion than the flexibility in the length direction, the connecting members 200 are disposed in a plurality of rows.

In addition, the disposition of the rows as well as the number of rows of the connecting member 200 can be changed according to types and positions of the tubules.

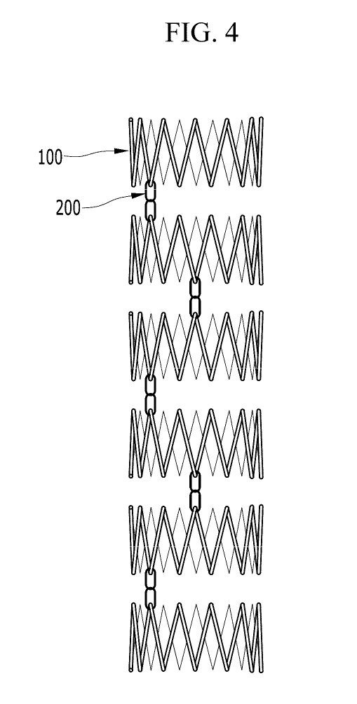

In a case that the tubule moves in one direction, the connecting members 200 are disposed in a linear row as shown in FIG. 1 and FIG. 2. In a case that the tubule moves in various directions, the connecting members 200 are disposed in a row of a zigzag shape as shown in FIG. 3 and FIG. 4.

In one or some embodiments, the connecting members 200 of the linear row are disposed along the length direction of the cylindrical member 100 or are disposed slanted in the length direction of the cylindrical member 100. In a case that the connecting members 200 are disposed slanted in the length direction of the cylindrical member 100, the shape of the stent can be changed more freely according to the movements of the tubules.

The shape of the rows of the connecting member 200 can be applied to the case where the connecting members 200 are disposed in a plurality of rows as well as the case where the connecting members 200 are disposed in one row.

That is, in a case where the connecting members 200 are disposed in a plurality of rows, each row can be disposed with a linear shape, a zigzag shape, or a combination of a linear shape and a zigzag shape.

Hereinafter, processes of inserting the stent according to exemplary embodiments of the present invention into the tubule and an operation of the stent will be described in detail referring to FIG. 6 to FIG. 8.

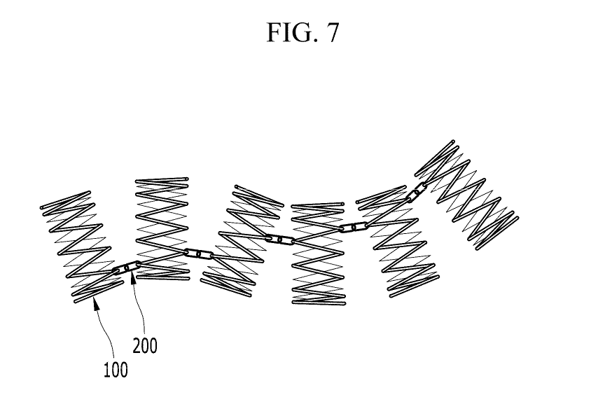

FIG. 6 and FIG. 7 are top plan views of a stent according to exemplary embodiments of the present invention for showing an operation of the stent, and

FIG. 8 is a top plan view of a stent according to another exemplary embodiment of the present invention for showing an operation of the stent.

In order to insert the stent according to exemplary embodiments of the present invention into the tubule, external force is applied radially inwardly to the cylindrical member 100 so as to decrease the diameter thereof. As described above, if the external force is applied radially inwardly to the cylindrical member 100, the angles α and β of the peak part 120 and the valley part 130 are decreased. Therefore, the diameter of the cylindrical member 100 is also decreased.

After that, the mounting position of each cylindrical member 100 is determined. At this time, the position of the connecting member 200 is also determined considering the movement of the tubule. After that, the cylindrical members 100 of which diameters are decreased are inserted into the tubule corresponding to the mounting position thereof, and the connecting member 200 is positioned at the target position.

In addition, the external force is removed from the cylindrical members 100 in a sequence from the cylindrical member 100 inserted deepest to the cylindrical member 100 inserted shallowest. In this case, the angles α and β of the peak part 120 and the valley part 130 of the cylindrical member 100 from which the external force is removed are increased again, and each cylindrical member 100 is mounted in the tubule. That is, the stent is completely mounted in the tubule. In one or some embodiments, the external force is removed from the cylindrical members 100 in a sequence from the cylindrical member 100 inserted shallowest to the cylindrical member 100 inserted deepest. In one or some embodiments, the cylindrical members 100 are sequentially mounted one after another regardless of the sequence.

If the stent is mounted in the tubule, the tubule expands and is no longer narrowed.

As described above, a plurality of cylindrical members 100 connected by the connecting member 200 can move relative to each other. Therefore, even if the movement of the tubule in which the stent according to exemplary embodiments of the present invention is mounted becomes bigger, the possibility of releasing the stent from the tubule is reduced. That is, each cylindrical member 100 can move freely corresponding to the movement of the tubule.

Meanwhile, a plurality of cylindrical members 100 in the stent that move freely to a certain degree are connected by the connecting member 200, and the connecting member 200 is elongated or shortened by the movements of the cylindrical members 100 according to the movement of the tubule. Therefore, the connecting member 200 can have reduced dynamic fatigue.

As described above, stress concentration is prevented and dynamic fatigue is reduced by forming the connecting member 200 with a ring-shape or connecting the plurality of ring-shape thin members in one or some embodiments.

In a case that a linear connecting member 200, for example, is used as shown in FIG. 8, stress is concentrated on the connecting member 200 by the movements of the cylindrical members 100 according to the movement of the tubule. In addition, in a case that stress is concentrated repeatedly, the connecting member 200 can fail due to fatigue. In order to prevent fatigue failure of the connecting member 200, the strength of the linear connecting member 200 is enhanced or the ring-shape connecting member 200 is used. Since a plurality of cylindrical members are connected by at least one connecting member according to exemplary embodiments of the present invention, flexibility in a length direction is maintained and durability is secured.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A stent comprising:
a plurality of cylindrical members having a hollow cylindrical shape having both ends open, and adapted to decrease a diameter thereof by an external force so as to be mounted in a tubule; and
a connecting member connecting cylindrical members confronting each other.

2. The stent of claim 1, wherein the cylindrical member is manufactured by disposing one or more lines in a zigzag shape on an external circumference thereof so as to form a plurality of peak parts and valley parts.

3. The stent of claim 2, wherein the connecting member connects the closest peak part and valley part of confronting cylindrical members.

4. The stent of claim 1, wherein the connecting member is manufactured by a linear thin member or a ring-shaped thin member.

5. The stent of claim 1, wherein the ring-shaped thin member is manufactured by cutting a thin pipe perpendicularly or slantedly to a length direction thereof.

6. The stent of claim 1, wherein the connecting member is disposed only on one semicircular portion in a case that the cylindrical member is divided by a plane passing through the center of the cylindrical member.

7. The stent of claim 6, wherein the connecting member is disposed in one row.

8. The stent of claim 7, wherein the row of the connecting member has a linear shape.

9. The stent of claim 7, wherein the row of the connecting member has a zigzag shape.

10. The stent of claim 6, wherein the connecting members are disposed in two or more rows.

11. The stent of claim 10, wherein the row of the connecting member has a linear shape, a zigzag shape, or a combination of a linear shape and a zigzag shape.

12. The stent of claim 1, wherein the cylindrical member is made of a harmless metal or resin material.

13. The stent of claim 1, wherein the connecting member is made of harmless metal or resin material.

14. The stent of claim 2, wherein the cylindrical member is adapted to change a diameter thereof by changing angles of the peak part and the valley part in a case that the external force is applied to the cylindrical member.

15. A method of mounting a stent having a plurality of cylindrical members adapted to decrease a diameter thereof by an external force, and a connecting member connecting cylindrical members confronting each other, comprising:
decreasing the diameter of the plurality of cylindrical members by exerting the external force thereon;
inserting the cylindrical members in a tubule;
mounting one of the cylindrical members at the tubule by removing the external force exerted on the one of the cylindrical members; and
sequentially mounting the others of the cylindrical members.

16. The method of claim 15, wherein the cylindrical members are mounted in a sequence from the cylindrical member inserted deepest to the cylindrical member inserted shallowest.

17. The method of claim 15, wherein the cylindrical members are mounted in a sequence from the cylindrical member inserted shallowest to the cylindrical member inserted deepest.

18. The method of claim 15, wherein the connecting member is adapted to move the plurality of cylindrical members relatively according to a movement of the tubule.
